(19) United States
(12) Patent Application Publication
(10) Pub. No.: US 2006/0212128 A1
(43) Pub. Date: Sep. 21, 2006

(54) Artificial limb assembly having microprocessor-controlled vacuum pump

(75) Inventor: Thure Nachbar, Gravois Mills, MO (US)

Correspondence Address: HOVEY WILLIAMS LLP, 2405 GRAND BLVD., SUITE 400, KANSAS CITY, MO, 64108, US

(73) Assignee: Jaems E. and Joyce E. Smith

(21) Appl. No.: 11/081,205

(22) Filed: Mar. 16, 2005

(51) Int. Cl. A61F 2/70; A61F 2/80

(52) U.S. Cl. 623/24; 623/34

(57) ABSTRACT

A vacuum-assist artificial limb assembly (10) is provided having a socket (22) for receiving a residual limb (20). The assembly (10) includes an on-board vacuum pump and control assembly (18) with a selectively operable vacuum pump (72) controlled by a microprocessor (44). The microprocessor (44) is also connected with an on-off switch (48), pressure adjust buttons (68), a pressure read-out (66), and an alarm (70). In use, a pressure transducer (74) in communication with the interior of socket (22) and coupled with microprocessor (44) monitors negative pressure conditions within the socket, and the microprocessor (44) operates pump (72) in response to transducer pressure signals. In this manner, the vacuum-assist operation of assembly (10) is essentially automatic.

### BACKGROUND OF THE INVENTION

1. Field of the Invention

The present invention is broadly concerned with improved prosthetic devices such as artificial limb assemblies of the type incorporating a vacuum pump in order to establish negative pressure conditions serving to securely attach the devices to residual limbs. More particularly, the invention is concerned with such prosthetic devices, and methods of operation thereof, wherein the devices include a vacuum-generating assembly including a powered vacuum source as well as a digital control assembly (e.g., a microprocessor) which is programmed to develop and maintain preselected negative pressure conditions.

2. Description of the Prior Art

An amputee losing part of an extremity or limb such as an arm or leg normally requires a prosthetic device such as an artificial limb to maintain optimum activity and functionality. The remainder of amputated limbs are commonly referred to as residual limbs, and these come in various sizes and shapes, which may vary over time. Many new amputations present residual limbs which are slightly bulbous or cylindrical in shape, whereas older amputations may have atrophied to a more conical shape. Residual limbs may also have individual problems owing to scarring, skin grafts, bony protuberances, uneven volume, neuroma, pain, or edema.

Broadly speaking, prosthetic limb assemblies provide a socket which is typically custom-manufactured for a particular residual limb, in order to ameliorate the problems outlined above. Also, a pylon or other elongate connector is secured to the socket and in turn supports a prosthetic foot or hand device. In recent years, artificial limb assemblies have made use of vacuum sources or pumps in order to generate negative pressure conditions serving to secure the socket to the residual limb. This type of connection has been found to be superior to prior devices using only mechanical connections such as straps.

For example, Jim Smith Sales, Inc. has distributed vacuum-type prosthetic devices under the Trademark TSS VACULINK. These devices include a vacuum pump which is motion-activated, e.g., as the user walks in the case of a prosthetic leg device, the walking motion and weight of the user provides the power needed to operate the vacuum pump. Other such devices are illustrated in U.S. Pat. Nos. 6,726,726, 6,761,642 and 5,549,709.

While prior motion or weight-operated vacuum prosthetic devices have achieved substantial success in the market place, they suffer from a number of drawbacks. First, during periods where the amputee is at rest, no vacuum can be generated. Thus, the user may experience a situation where the device becomes loose or even detaches from the residual limb, owing to inactivity over a period of time. Additionally, there is generally no way to periodically or continuously monitor the actual negative pressure conditions within the socket, so that the magnitude of negative pressure may vary over wide limits. It is also generally known that residual limbs tend to lose volume over the course of the day if the negative pressure within the socket decreases beyond a certain threshold. This can be a problem during periods of rest in these weight or motion operated devices. Finally, these prior motion or weight-activated devices are limited to particular applications such as specific types or brands of prosthetic components and certain residual limb lengths.

Accordingly, there is a need in the art for improved vacuum-type prosthetic devices which overcome the problems inherent in prior devices and are operable to establish and maintain negative pressure conditions on an essentially automatic basis regardless of the degree of activity of the user.

### SUMMARY OF THE INVENTION

The present invention overcomes the problems outlined above and provides improved prosthetic devices such as artificial limbs. These devices have an electrically-powered on-board vacuum pump controlled by a digital controller such as a microprocessor. Broadly speaking, the artificial limb assemblies of the invention include a socket for receiving a residual limb and a vacuum source operatively coupled with the socket in order to generate a negative pressure therein; additionally, the assemblies have a digital control assembly coupled with the vacuum source and operable to control the operation thereof in order to maintain sufficient and consistent negative pressure within the socket to keep the limb assembly in place on the residual limb. The socket is preferably a hypobarically controlled prosthetic socket and the vacuum source is preferably a dual diaphragm, rechargeable battery or battery powered, microprocessor-controlled vacuum pump capable of maintaining a high level of negative pressure in the socket.

The preferred digital control assemblies of the invention include user-operated structure for adjusting the output of the vacuum source for adjusting the level of negative pressure within the socket. In this way, maximum comfort and operational flexibility can be obtained. These effects are enhanced by means of a read-out device forming a part of the control assembly for displaying the negative pressure conditions within the socket. Preferably, the entire vacuum pump and control assembly is self-contained and mounted on the artificial limb, such as on the upright pylon of an artificial leg assembly. Optionally, a perceptible alarm may also be included which will give an alarm signal (e.g., audible or visual) if the battery fails or is low. In preferred forms, the read out device will be able to display a variety of information selected from the group consisting of current vacuum pressure within the socket, the set point of the maximum and minimum vacuum pressures to be drawn in the socket, and remaining battery life.

Digital control of the vacuum pump is achieved by using the digital controller to periodically or essentially continuously monitor vacuum conditions within the socket. To this end, a pressure transducer is preferably coupled in communication with the interior of the socket and delivers pressure signals to the digital controller; the latter initiates or terminates pump operation in response to such pressure signals. Preferably, the range of pressures to be maintained will be able to be programmed on each individual pump unit. For example, some individuals will prefer to have a negative pressure variation of 1 inch of mercury or less while others will prefer a wider range. However, it is understood that the invention herein is capable of all such types of variation.

In one preferred embodiment of the present invention, the invention includes a socket assembly, a flexible liner, and a vacuum pump and control assembly. The flexible liner is preferably a synthetic resin sock such as a conventional urethane liner adapted to snugly fit over a residual limb. The socket assembly generally includes an upright, open-top socket having a closed lower end adapted to receive and attach to a prosthetic limb. The open top of the socket receives the residual limb and liner therein. An opening adapted to receive a vacuum hose is also present on the socket assembly and this opening fluidly connects the exterior of the socket assembly with the interior. In some preferred forms, the opening is a threaded bore or is adapted to receive a conventional barb connector therein. A vacuum hose connects the opening with the vacuum pump and control assembly. Initiation of a pump cycle begins when the digital control responds to a pressure signal below the minimum threshold set by the user. The vacuum generated by the pump draws the liner to the socket and the residual limb to the liner, thereby providing a secure fit, a decrease (or elimination) of gaps between the residual limb, liner, and socket, consistent negative pressure within the socket assembly, and a decrease in residual limb volume loss.

In other preferred forms, the invention is coupled with a conventional prosthetic pylon and appendage such as a hand or foot. Advantageously, the socket assembly of the present invention is not limited to any particular prosthetic component and does not require any particular stump length or size. Accordingly, it is universally adaptable to a wide variety of currently existing applications.

The vacuum pump and control assembly is preferably secured to the prosthetic device using any conventional means including tape, elastic bands, screws, bolts, hook and loop wraps or straps, custom designed pockets, clips, and the like. However, it is understood that the vacuum pump and control assembly may also be secured to a location remote from the prosthetic such as on or in the socket assembly (e.g. in a custom container mounted on the socket) or even to the individual.

Preferred forms of the invention may also include a sealing means adapted to maintain separation between the interior of the socket assembly to which the vacuum pressure is applied and the outside atmosphere. This can be accomplished using a variety of means including customized synthetic resin sleeves, conventional sealing sleeves, tape, and elastic bands. A good sealing means will decrease the number of pump cycles the vacuum pump will initiate.

### BRIEF DESCRIPTION OF THE DRAWINGS

FIG. 1 is a side view partially in phantom of an artificial limb assembly in accordance with the invention, shown mounted upon a residual limb;

FIG. 2 is an enlarged view partially in vertical section depicting the socket assembly forming a part of the artificial limb assembly; and

FIG. 3 is a schematic representation of the vacuum pump and control assembly of the artificial limb assembly.

### DETAILED DESCRIPTION OF THE PREFERRED EMBODIMENT

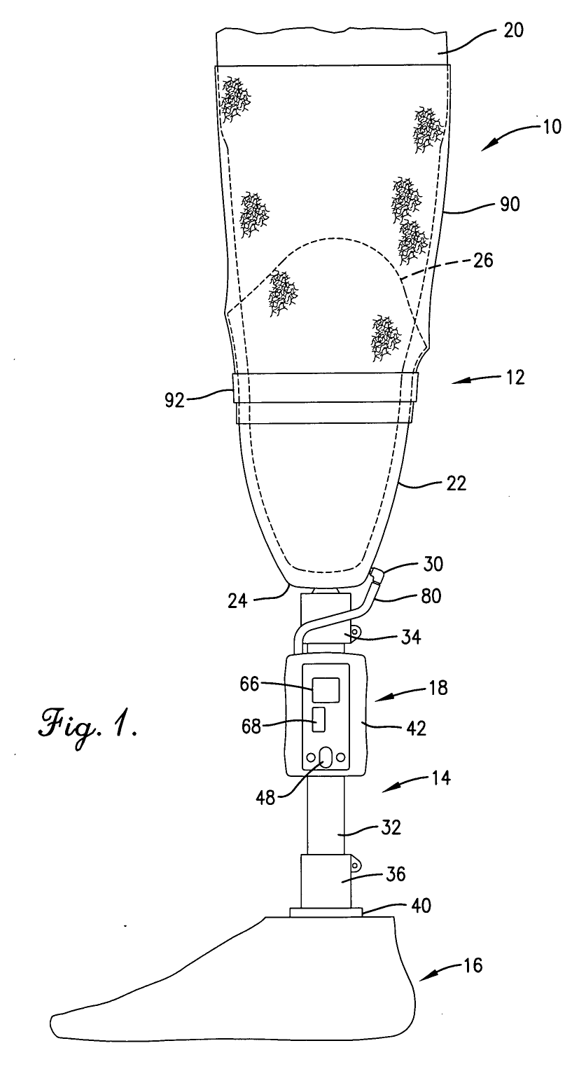

Turning now to the drawings, an artificial limb assembly 10 is depicted in FIG. 1 and broadly includes a socket assembly 12, a pylon 14, a prosthetic foot 16, and a vacuum pump and control assembly 18. The limb assembly 10 is adapted to be coupled with a residual limb 20, in this case, the residuum of a below-the-knee amputation. It will be appreciated, however, that the invention is not limited to this specific type of artificial limb assembly, but can be used for other varieties, e.g., above-the-knee amputations or for artificial arm assemblies.

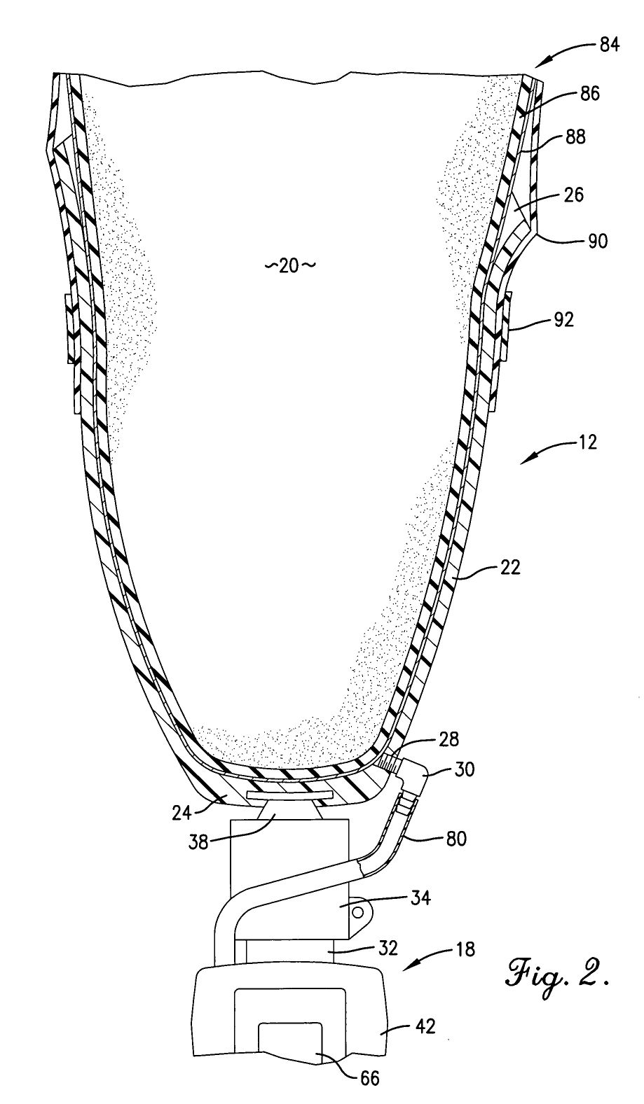

The socket assembly 12 is best illustrated in FIG. 2, where it will be seen that it includes an upright, open-top relatively rigid socket 22 presenting a lower closed-end 24 and an upper margin 26. It will also be seen that the socket 12 includes a threaded bore 28 receiving a threaded pneumatic nipple 30, which is important for purposes to be described. Generally, the socket 22 would be custom-prepared for an individual patient, in order to best accommodate the residual limb 20.

The pylon 14 is itself conventional and includes a primary aluminum and/or composite rod 32 having endmost upper and lower clamps 34 and 36. The upper clamp 34 includes a socket adapter 38 which is received within the body of socket 22 at end 24, and serves to provide an appropriate connection between socket 22 and pylon 14. Similarly, lower clamp 36 has an adaptor 40 affording a connection to prosthetic foot 16.

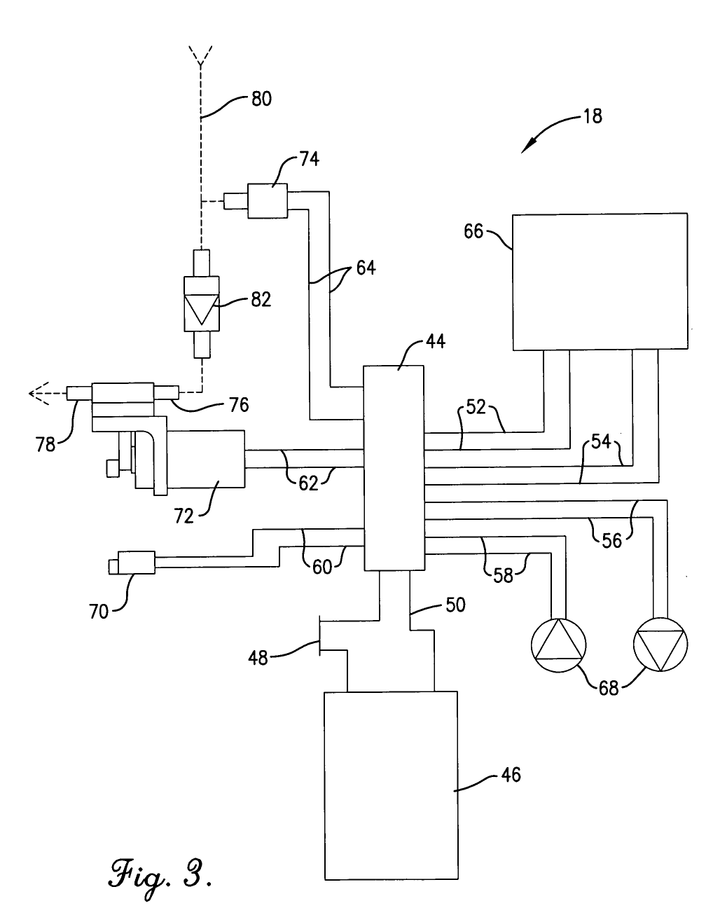

The vacuum pump and control assembly 18 is located within a housing 42 secured to pylon rod 32 by any conventional means such as those described previously. Generally speaking, the function of assembly 18 is to create negative pressure conditions within socket 22 and to maintain these conditions within predetermined limits. Attention is directed to FIG. 3 which depicts the components of assembly 18. Specifically, assembly 18 includes a digital microprocessor controller 44 powered by a battery 46 and with an on-off switch 48 located in the leads 50 between the controller and battery. The microprocessor 44 is operatively connected to a number of components via leads 52, 54, 56, 58, 60, 62, and 64. Specifically, the leads 52, 54 are coupled to a conventional pressure display 66 while leads 56 and 58 are coupled to manual pressure adjust buttons 68. The leads 60 are connected with a vibratory alarm 70 while leads 62 are coupled to a vacuum pump 72. Finally, the leads 64 are connected with a pressure transducer 74.

The pump 72 includes a vacuum inlet 76 as well as an opposed venting outlet 78. A flexible vacuum line 80 extends between inlet 76 and nipple 30 and is equipped with a check valve 82. If desired, an in-line filter (not shown) may be installed in the line 80 between the pump and the socket in order to filter small particles, lint and dust and to prevent these from entering the pump. As illustrated, the transducer 74 is in communication with line 80 upstream of check valve 82.

In use, the residual limb 20 is first inserted with socket 22. Normally, the residual limb is covered by a pliable synthetic resin sock or liner 84 having a resilient layer 86 and an outer layer 88. An optional synthetic resin sleeve 90 may be applied over the juncture between the residual limb 20 and upper margin 26 of socket 22, with the sleeve 90 being held in place by elastic band 92.

Next, the assembly 18 is used to create negative pressure conditions within socket 22 serving to hold residual limb 20 in place therein. This involves actuating on-off switch 48 which, through microprocessor 44, initiates operation of vacuum pump 72 to generate a predetermined vacuum pressure within the socket 22. As manufactured, the controller 44 would typically be set to establish and maintain negative pressure conditions of between 10-15 inches of mercury, which has been found to maintain optimal suspension and residual limb control during normal activity. The practical limit of negative pressure is around 22-25 inches of mercury, these being the maximum levels the pump will achieve for extremely high activity levels, such as patient's competing in sports. However, the nominal level can readily be changed by manipulation of the pressure adjust buttons 68. The pressure transducer 74 measures the negative pressure conditions within line 80 and thus socket 22, and the microprocessor 44 uses the transducer output to control the operation upon vacuum pump 72. This occurs not only during initial start-up, but periodically or even essentially continuously while the assembly 10 is being worn. Thus, if the negative pressure conditions within socket 22 reach a point outside of the predetermined, selected range for the user, the microprocessor 44 initiates operation of pump 72 as needed. For example, a given user may select a range of 12-19 inches of mercury. When the vacuum conditions within socket 22 bleed down to 12 inches or below, the pump 72 is actuated to return the vacuum level to the desired 19 inches of mercury, whereupon the pump operation is terminated. Also, as a separate safety measure, the alarm 70 may be actuated in the event of any out of specification pressure conditions, to generate a perceptible alarm signal such an audible or vibratory signal.

The invention provides a number of advantages not heretofore possible with vacuum-type artificial limb assemblies. Use of the microprocessor 44 in assembly 18 permits assembly automatic operation which can be readily programmed to achieve and maintain a desired vacuum condition. The invention does not rely upon any weight or motion-activation, which can be problematic during periods of patient rest or where there are patient limits on the use of such equipment. Moreover, there is no practical patient weight limitation because of the non-structural usage of the invention. Rather, any patient weight limit for the prosthesis is determined by the prosthetic components selected.

I/we claim:

1. In an artificial limb assembly including a socket for receiving a residual limb, and a vacuum source operatively coupled with the socket in order to generate a negative pressure therein, the improvement which comprises a digital control assembly coupled with said vacuum source and operable to control the operation thereof in order to maintain sufficient negative pressure within said socket to keep the limb assembly in place on said residual limb.

2. The assembly of claim 1, said limb assembly including a pylon secured to said socket and a prosthetic foot supported by the pylon.

3. The assembly of claim 1, said digital control assembly including a microprocessor coupled with said vacuum source.

4. The assembly of claim 1, said digital control assembly including structure for adjusting the output of said vacuum source for adjusting the level of negative pressure within said socket.

5. The assembly of claim 1, said digital control assembly including a read-out device for displaying the negative pressure conditions within said socket.

6. The assembly of claim 1, said artificial limb assembly including a pylon, said vacuum source and digital control assembly being supported on said pylon.

7. The assembly of claim 1, said digital control assembly including a perceptible alarm operable to emit an alarm signal in the event that negative pressure conditions within said socket are determined to be outside a predetermined limit.

8. The assembly of claim 1, including a sealing sleeve disposed about the upper end of said socket.

9. A method of attaching and maintaining an artificial limb assembly to a residual limb, said artificial limb assembly including a socket and a vacuum source operatively coupled with said socket in order to generate a negative pressure within the socket, said method comprising the steps of:
inserting said residual limb into said socket and operating said vacuum source in order to generate negative pressure conditions therein serving to attach the socket to said residual limb; and
using a digital controller to periodically monitor the negative pressure conditions within said socket, and to operate said vacuum source as necessary to maintain sufficient negative pressure within the socket to keep the limb assembly on said residual limb.

10. The method of claim 9, including the step of continuously monitoring said negative pressure conditions within said socket.

11. The method of claim 9, including the step of using a microprocessor as said digital controller.

12. The method of claim 1, including the step of generating a perceptible alarm signal in the event that the negative pressure conditions within said socket fall outside of a predetermined limit.

13. An assembly for maintaining negative pressure within a prosthetic socket for a residual limb comprising:
a flexible liner adapted to receive a residual limb therein and having an interior face adjacent the residual limb and an exterior face;
a socket adapted to receive said flexible liner and said residual limb therein, said socket having an interior surface adjacent said exterior face of said flexible liner;
a sealer separating the interior surface of said socket from ambient air pressure; and
a vacuum pump adapted to maintain a negative pressure within the socket.

14. The assembly of claim 13, said vacuum pump including a digital control assembly having a microprocessor for monitoring and maintaining said negative pressure.

15. The assembly of claim 14, said microprocessor adapted to cycle said vacuum pump on and off in response to pressure conditions within said socket.

16. The assembly of claim 13, said vacuum pump including structure for adjusting the amount of vacuum pressure to be maintained in said socket.

17. The assembly of claim 13, further comprising a prosthetic limb or appendage connected to said socket.
